(19) United States
(12) Patent Application Publication
(10) Pub. No.: US 2004/0028583 A1
(43) Pub. Date: Feb. 12, 2004

(54) Portable decontamination unit useful in destroying harmful biological agents in contaminated objects

(76) Inventor: David E. Hedman, Ojai, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/313,901

(22) Filed: Dec. 5, 2002

Related U.S. Application Data
(60) Provisional application No. 60/339,184, filed on Dec. 7, 2001.

(51) Int. Cl. A62B 7/08; A61L 2/00; A61L 9/00

(52) U.S. Cl. 422/292; 422/123; 422/305; 422/307

(57) ABSTRACT

A portable decontamination unit includes a portable housing which defines an interior sealable cavity accessible through a door. An inlet and an outlet are provided through the housing for introducing heated air into and removing the heated air from the cavity. A temperature sensor is positioned within the cavity for monitoring the temperature of the cavity and its contents, and an electronic display is provided of the temperature within the cavity over time. An ultraviolet lamp is positioned within the cavity for irradiating objects to be decontaminated with ultraviolet light. An air scrubber is associated with the outlet, and fans are associated with both the inlet and outlet to condition air being forced into and extracted from the cavity.

### RELATED APPLICATION

This application claims priority from U.S. Provisional Application Serial No. 60/339,184, filed Dec. 7, 2001.

### BACKGROUND OF THE INVENTION

The present invention generally relates to devices for sanitizing and decontaminating objects. More particularly, the present invention relates to an apparatus for killing and removing bacteria, mold, viruses, and other harmful organic substances from objects which have become contaminated, or which may be contaminated.

Very recently the threat of bioterrorism has been realized in that letters, other packages, and/or personal items containing anthrax have been sent to high profile media personnel and government officials. Many postal workers, mail room clerks, secretaries and others have become infected, and some have even died due to the exposure of the anthrax from such packages. Unfortunately, it has been found that not only those coming into contact with the package itself, but others that are in the proximity of the package have become infected as the dust or residue on or within the package has leaked and contaminated other objects and areas around the package. Understandably, there has been a great concern and need to decontaminate letters, packaging or personal items delivered through the mail, and in the event that a building has been contaminated, the decontamination of objects within the building to prevent further exposure and disease caused by such biological agents or allergens.

Thus, there is a need for a portable apparatus for treating letters and packages delivered through the mail or received by courier, and for decontaminating other objects within a structure which may otherwise be difficult to decontaminate utilizing standard chemical and biohazard decontamination techniques. It will be appreciated that if the letters and packages are first decontaminated or treated before being delivered, the risk of exposure to anthrax or other bioterrorism agents will be significantly reduced. The present invention fulfills these needs and provides other related advantages.

### BRIEF DESCRIPTION OF THE DRAWINGS

The accompanying drawings illustrate the invention. In such drawings:

FIG. 1 is a front elevational view of a portable decontamination unit employed in the present invention, wherein a front wall having doors has been removed to show the interior of the decontamination unit; and

FIG. 2 is a side elevational view of the portable decontamination unit of FIG. 1.

### SUMMARY OF THE INVENTION

The present invention resides in a portable decontamination unit useful in destroying harmful biological agents in contaminated objects. The portable decontamination unit of the present invention permits a large quantity of mail and other objects to be effectively treated in a relatively short amount of time, all within a unit that can be moved to locations where it is needed most.

In a preferred form of the invention, the portable decontamination unit comprises a portable housing which defines an interior sealable cavity accessible through a door. An inlet is provided for introducing heated air into the cavity, and an outlet is provided for removing air from the cavity. A temperature sensor is provided within the cavity for monitoring the temperature of the cavity and its contents. Further, an electronic display permits monitoring of the temperature within the cavity over time.

In one form of the invention, wheels are associated with the housing to facilitate movement of the decontamination unit from one location to another. A perforated baffle is disposed within the cavity of the housing through which heated air passes. An ultraviolet lamp is also positioned within the cavity for irradiating objects to be decontaminated with ultraviolet light. Moreover, the electronic display includes a printer.

Means are provided for supporting objects to be decontaminated within the cavity of the housing. The supporting means may comprise a shelf which includes a pair of oppositely facing brackets fixed to interior surfaces of the housing. Further, the supporting means may include a rod within the cavity which enables the hanging of objects thereon.

Means are associated with the inlet, for introducing heated air into the cavity under positive pressure. Such introducing means may comprise a fan or similar device. Means are also associated with the outlet, for extracting air from the cavity. The extracting means may comprise an exhaust fan. Further, an air scrubber, such as high efficiency particulate arrestance (HEPA) and carbon filters, is provided to remove the remains of biologic organisms from exhaust air and to prevent them from reaching the environment.

Other features and advantages of the present invention will become apparent from the following more detailed description, taken in conjunction with the accompanying drawings which illustrate, by way of example, the principles of the invention.

### DETAILED DESCRIPTION OF THE PREFERRED EMBODIMENT

As shown in the drawings for purposes of illustration, the present invention resides in a portable decontamination unit, generally referred to by the reference number 10, which destroys harmful organic agents such as microbiological, bacterial and viral agents, from objects which have been contaminated.

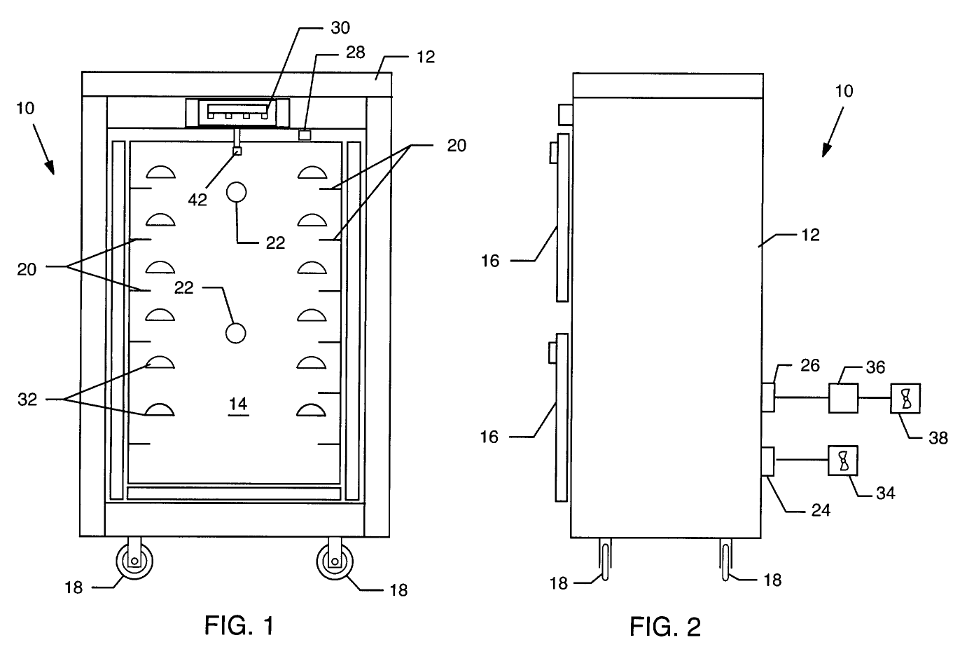

With reference to FIGS. 1 and 2, there is shown an exemplary portable decontamination unit 10 having a housing 12 which defines an interior cavity 14 accessible through one or more doors 16. Preferably, the housing 12 is comprised of stainless steel so as to be durable, capable of withstanding high temperatures, as well as capable of being sanitized quite easily. The unit 10 shown is 31.625 inches wide, 71.187 inches tall, and 33.00 inches deep. It should be understood, however, that the unit 10 can be of various sizes, configurations and portability. Although the edges of the illustrated unit are relatively square and sharp, preferably the edges of the unit 10 are rounded to provide greater safety to the users thereof.

The unit 10 shown is portable, and as such includes wheeled casters 18 to facilitate movement of the unit 10. The unit 10 may be incorporated into a trailer, or it may also be built in a fixed unit configuration.

The housing 12 and doors 16 cooperatively seal the enclosure 14. High quality gaskets are installed around the doors 16 in the unit 10 to control the ingress and egress of gas (air) with a complete and secure seal. This renders the unit 10 safer and more efficient to use by not allowing heat or biologics to escape and contaminate the surrounding environment. Preferably, a glass window is installed in each door 16 to observe the contents during the heating cycle process. This allows the operator to monitor the condition of the contents.

The interior of the housing includes several “L” brackets 20 which serve as shelves for placing trays of objects therein, such as trays of letters, packages, etc. Tubes or rods 22 extend into the cavity 14 to enable the hanging of other objects therein. Preferably, fans are installed inside of the unit 10 to create air turbulence. This forced movement of air in the unit 10 may increase the heat penetrative capability of the process and provide for even more heating. Also, means for introducing humidity into the cavity 14, such as a nebulizer, bath of water in the base of the unit 10, etc. is preferably provided as moisture can provide greater penetrative capability for certain biological contaminants. The addition of humidity may also allow the inactivation of contaminants at lower temperatures.

The unit 10 includes inlet and outlet ducts 24 and 26 for introducing and removing air. The unit 10 may include an internal heater, or heated air may be introduced through inlet 24 and removed from outlet 26. In some applications, a cooler may be included to more quickly cycle contents of the unit 10 through the decontamination procedure described below, and to help maintain an acceptable ambient temperature around the unit 10.

Most bacteria are destroyed at 130° F. after being exposed to this temperature for thirty minutes or more. However, it has been found that the anthrax spore requires temperatures in excess of 248° F. for thirty minutes or more in order to be completely destroyed. The Department of Defense of the United States has identified the thirteen most probable bio-hazardous organisms and materials. All of them can be neutralized within the portable decontamination unit 10 of the present invention, at an internal temperature of 320° F. for 8-12 hours. Air is heated to a temperature that is lethal to the organism to be destroyed. Thus, for complete disinfection, the air temperature is raised to at least 248° F. to kill the anthrax spores. It has been found that the flash point of letters and packaging is in excess of 400° F. Objects, such as mail, personal belongings, etc., placed within the cavity 14 will not be significantly damaged by temperatures well below their flashpoint.

The unit 10 includes one or more temperature sensors and/or probes 28, (fixed or portable) to monitor the internal temperature of the cavity 14 and its contents. A display 30 informs the user of the unit 10 of the temperature, time, etc. It is contemplated that temperature probes 28 could be used and placed within the letters and packaging to ensure that the proper temperature is attained throughout all of the objects to be decontaminated. Such probes could be wired into the display 30. Preferably, the display 30 comprises a digital readout that is constructed into the unit 10. This display 30 may include a printer, or be connected to a printer, for printing out the internal cavity 14 temperature over time. This provides users of the unit 10 documentation of maintaining the lethal temperature for the required time.

In use, objects to be decontaminated, such as mail, are placed within the cavity 14 and the doors 16 locked so as to seal the cavity 14. Air heated in excess of 248° F. is introduced through the inlet 24 with a positive air pressure using a blower or the like. Ozone may be introduced into the cavity 14 with the heated air. The air enters into the cavity 14 through a perforated baffle 32. Preferably, the air is circulated using fans 34 or the like, although due to the size of the unit 10 the introduction of the air through the inlet 24 is probably sufficient to ensure an overall consistent temperature within the cavity 14. The heated air may be passed through an air scrubber 36, such as high efficiency particulate arrestance (HEPA) and carbon filters, to remove the remains of the organisms from the air and to prevent them from reaching the environment. Other filters 36 such as charcoal filters or UV filters may be employed as well. It has been found that mail gives off volatile organic compounds (VOC), which can be toxic. Also, certain biologic bodies may pose a health risk, in the form of allergens, even when inactivated. The VOC's given off by plastic components and resins of the mail, and inactivated biological bodies will be removed by a filtration system 36. For optimum effectiveness, it is often desirable to increase air flow rates by adding a vacuum device 38, such as a blower, in association with the air scrubber to aid in the extracting of the air from the unit 10. The force of extraction also increases the efficiency of filtering of the air by the air scrubber. Heated air may be directed into the unit 10 faster than it is exhausted, thereby creating a positive pressure inside the unit 10. This positive pressure, in addition to heat, kills the organisms within the unit 10. Alternatively, a negative pressure may be applied to the unit 10. Airflow ports may be installed in the unit 10 to control or make up air for negative air flow into the cavity 14. This provides the operator of the unit 10 greater control over heat and negative air flow.

Some organisms, such as anthrax spores, can be particularly difficult to completely destroy. Accordingly, the unit 10 preferably includes one or more ultraviolet light fixtures so that ultraviolet lamps 42 can direct ultraviolet light onto the objects to be decontaminated. It is well known that such ultraviolet light can serve to kill harmful organisms. In addition, biocides, fumigants or ozone may be injected or placed into the cavity 14, such as through the inlet 24, which synergistically react with the heated air to destroy the organisms.

After treating the objects for the predetermined temperature and time, the air is removed through the outlet 26 under negative pressure in order to facilitate the dislodgement of the contaminants from the objects and filtering them from the air which is discharged from the unit 10. In some instances, quickly cooling objects placed within the cavity 14 may desirably further decontaminate objects within the housing 12. This is accomplished by cooling the cavity 14 quickly from an elevated temperature so that the target microbiological, bacterial and/or viral agents are unable to adjust to the change in temperature, thereby neutralizing them.

It will be appreciated that a large quantity of mail and other objects can be effectively treated in a relatively short amount of time using the unit 10 of the present invention. The invention can be used not only to decontaminate objects which have been known to be contaminated, but can also be used as a preventive measure in treating, for example, mail before it is delivered to avoid the possibility of contamination.

Although an embodiment has been described in detail for purposes of illustration, various modifications may be made without departing from the scope and spirit of the invention. Accordingly, the invention is not to be limited, except as defined in the following claims.

What is claimed is:

1. A portable decontamination unit useful in destroying harmful biological agents in contaminated objects, the decontamination unit comprising:
a portable housing which defines an interior sealable cavity accessible through a door;
an inlet for introducing heated air into the cavity;
an outlet for removing air from the cavity;
a temperature sensor within the cavity for monitoring the temperature of the cavity and its contents; and
an electronic display of the temperature within the cavity over time.

2. The portable decontamination unit of claim 1, including wheels associated with the housing to facilitate movement of the decontamination unit.

3. The portable decontamination unit of claim 1, including a perforated baffle through which heated air passes within the cavity of the housing.

4. The portable decontamination unit of claim 1, including means for supporting objects to be decontaminated within the cavity of the housing.

5. The portable decontamination unit of claim 4, wherein the supporting means comprises a shelf including a pair of oppositely facing brackets fixed to interior surfaces of the housing.

6. The portable decontamination unit of claim 4, wherein the supporting means includes a rod within the cavity to enable the hanging of objects therein.

7. The portable decontamination unit of claim 1, wherein the electronic display includes a printer.

8. The portable decontamination unit of claim 1, including an ultraviolet lamp within the cavity for irradiating objects to be decontaminated with ultraviolet light.

9. The portable decontamination unit of claim 1, including means associated with the inlet, for introducing heated air into the cavity under positive pressure.

10. The portable decontamination unit of claim 9, wherein the introducing means comprises a fan.

11. The portable decontamination unit of claim 1, including means associated with the outlet, for extracting air from the cavity.

12. The portable decontamination unit of claim 11, wherein the extracting means comprises an exhaust fan.

13. The portable decontamination unit of claim 1, including an air scrubber associated with the outlet.

14. A portable decontamination unit useful in destroying harmful biological agents in contaminated objects, the decontamination unit comprising:
a portable housing which defines an interior sealable cavity accessible through a door;
a perforated baffle through which heated air passes within the cavity of the housing;
an inlet for introducing heated air into the cavity;
an outlet for removing air from the cavity;
an air scrubber associated with the outlet; and
a temperature sensor within the cavity for monitoring the temperature of the cavity and its contents.

15. The portable decontamination unit of claim 14, including an electronic display of the temperature within the cavity over time, the display including a printer.

16. The portable decontamination unit of claim 14, including wheels associated with the housing to facilitate movement of the decontamination unit.

17. The portable decontamination unit of claim 14, including a shelf for supporting a tray of objects to be decontaminated within the cavity of the housing, wherein the shelf comprises a pair of oppositely facing brackets fixed to interior surfaces of the housing.

18. The portable decontamination unit of claim 17, including a rod within the cavity to enable the hanging of objects therein.

19. The portable decontamination unit of claim 14, including an ultraviolet lamp within the cavity for irradiating objects to be decontaminated with ultraviolet light.

20. The portable decontamination unit of claim 14, including means associated with the inlet, for introducing heated air into the cavity under positive pressure.

21. The portable decontamination unit of claim 20, including means associated with the outlet, for extracting air from the cavity.

22. A portable decontamination unit useful in destroying harmful biological agents in contaminated objects, the decontamination unit comprising:
a portable housing which defines an interior sealable cavity accessible through a door;
an inlet for introducing heated air into the cavity;
means associated with the inlet for introducing heated air into the cavity under positive pressure;
an outlet for removing air from the cavity;
means associated with the outlet, for extracting air from the cavity;
an air scrubber associated with the outlet;
a temperature sensor within the cavity for monitoring the temperature of the cavity and its contents;
an ultraviolet lamp within the cavity for irradiating objects to be decontaminated with ultraviolet light;
a perforated baffle through which heated air passes within the cavity of the housing;
wheels associated with the housing to facilitate movement of the decontamination unit; and
an electronic display of the temperature within the cavity overtime, the electronic display including a printer.

23. The portable decontamination unit of claim 22, including means for supporting objects to be decontaminated within the cavity of the housing.
